(19) United States
(12) Patent Application Publication
(10) Pub. No.: US 2005/0192526 A1
(43) Pub. Date: Sep. 1, 2005

(54) Devices for maintaining surgically created openings

(76) Inventors: Michael Biggs, Santa Clara, CA (US); Thomas Keast, Sunnyvale, CA (US); Bryan Loomas, Los Gatos, CA (US); Don Tanaka, Saratoga, CA (US); David Thompson, San Jose, CA (US); Gary Kaplan, San Francisco, CA (US); Kelly Shriner, Arlington, MA (US); Halil Karabey, San Jose, CA (US); Russ Redmond, Goleta, CA (US); Claude Vida, Sanata Barbara, CA (US); Mike Collinson, Goleta, CA (US); Cary Cole, Mountain View, CA (US); Michael Willink, San Jose, CA (US)

Correspondence Address: BRONCUS TECHNOLOGIES, INC.;BUILDING A8, 1400 N. SHORELINE BLVD., MOUNTAIN VIEW, CA, 94043, US

(21) Appl. No.: 11/006,362

(22) Filed: Dec. 7, 2004

Related U.S. Application Data
(63) Continuation of application No. 10/951,962, filed on Sep. 28, 2004.
Application No. 10/951,962 is a continuation of application No. PCT/US03/12323, filed on Apr. 21, 2003.
(60) Provisional application No. 60/374,022, filed on Apr. 19, 2002.
(60) Provisional application No. 60/387,163, filed on Jun. 7, 2002.
(60) Provisional application No. 60/393,629, filed on Jul. 3, 2002.

(51) Int. Cl. A61F 2/04

(52) U.S. Cl. 604/8; 623/23.65; 623/23.7

(57) ABSTRACT

Devices and methods are directed to improving the gaseous exchange in a lung of an individual having, for instance, chronic obstructive pulmonary disease. More particularly, conduits may be deployed in the lung to maintain collateral openings (or channels) surgically created through airway walls. This tends to facilitate both the exchange of oxygen ultimately into the blood and decompress hyper-inflated lungs.

### CROSS REFERENCE TO RELATED APPLICATIONS

This is a continuation of U.S. patent application Ser. No. 10/951,962 filed on Sep. 28, 2004 which is a continuation of international application No. PCT/US03/12323 filed on Apr. 21, 2003, which is a non-provisional of U.S. provisional patent application No. 60/374,022 filed on Apr. 19, 2002, and a non-provisional of U.S. provisional patent application No. 60/387,163 filed on Jun. 7, 2002, and a non-provisional of U.S. provisional patent application No. 60/393,629 filed on Jul. 3, 2002. The entirety of all of the above listed applications are hereby incorporated by reference.

### BACKGROUND OF THE INVENTION

In 1995, the American Lung Association (ALA) estimated that between 15-16 million Americans suffered from chronic obstructive pulmonary disease (COPD) which includes diseases such as chronic bronchitis, emphysema, and some types of asthma. The ALA estimated that COPD was the fourth-ranking cause of death in the U.S. The ALA estimates that the rate of emphysema is 7.6 per thousand population, and the rate for chronic bronchitis is 55.7 per thousand population.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

To understand the detrimental effects of COPD, the workings of the lungs requires a cursory discussion. The primary function of the lungs is to permit the exchange of two gasses by removing carbon dioxide from arterial blood and replacing it with oxygen. Thus, to facilitate this exchange, the lungs provide a blood gas interface. The oxygen and carbon dioxide move between the gas (air) and blood by diffusion. This diffusion is possible since the blood is delivered to one side of the blood-gas interface via small blood vessels (capillaries). The capillaries are wrapped around numerous air sacs called alveoli which function as the blood-gas interface. A typical human lung contains about 300 million alveoli.

The air is brought to the other side of this blood-gas interface by a natural respiratory airway, hereafter referred to as a natural airway or airway, consisting of branching tubes which become narrower, shorter, and more numerous as they penetrate deeper into the lung. Specifically, the airway begins with the trachea which branches into the left and right bronchi which divide into lobar, then segmental bronchi. Ultimately, the branching continues down to the terminal bronchioles which lead to the alveoli. Plates of cartilage may be found as part of the walls throughout most of the airway from the trachea to the bronchi. The cartilage plates become less prevalent as the airways branch. Eventually, in the last generations of the bronchi, the cartilage plates are found only at the branching points. The bronchi and bronchioles may be distinguished as the bronchi lie proximal to the last plate of cartilage found along the airway, while the bronchiole lies distal to the last plate of cartilage. The bronchioles are the smallest airways that do not contain alveoli. The function of the bronchi and bronchioles is to provide conducting airways that lead air to and from the gas-blood interface. However, these conducting airways do not take part in gas exchange because they do not contain alveoli. Rather, the gas exchange takes place in the alveoli which are found in the distal most end of the airways.

The mechanics of breathing include the lungs, the rib cage, the diaphragm and abdominal wall. During inspiration, inspiratory muscles contract increasing the volume of the chest cavity. As a result of the expansion of the chest cavity, the pleural pressure, the pressure within the chest cavity, becomes sub-atmospheric. Consequently, air flows into the lungs and the lungs expand. During unforced expiration, the inspiratory muscles relax and the lungs begin to recoil and reduce in size. The lungs recoil because they contain elastic fibers that allow for expansion, as the lungs inflate, and relaxation, as the lungs deflate, with each breath. This characteristic is called elastic recoil. The recoil of the lungs causes alveolar pressure to exceed atmospheric pressure causing air to flow out of the lungs and deflate the lungs. If the lungs' ability to recoil is damaged, the lungs cannot contract and reduce in size from their inflated state. As a result, the lungs cannot evacuate all of the inspired air.

In addition to elastic recoil, the lungs' elastic fibers also assist in keeping small airways open during the exhalation cycle. This effect is also known as “tethering” of the airways. Such tethering is desirable since small airways do not contain cartilage that would otherwise provide structural rigidity for these airways. Without tethering, and in the absence of structural rigidity, the small airways collapse during exhalation and prevent air from exiting thereby trapping air within the lung.

Emphysema is characterized by irreversible biochemical destruction of the alveolar walls that contain the elastic fibers, called elastin, described above. The destruction of the alveolar walls results in a dual problem of reduction of elastic recoil and the loss of tethering of the airways. Unfortunately for the individual suffering from emphysema, these two problems combine to result in extreme hyperinflation (air trapping) of the lung and an inability of the person to exhale. In this situation, the individual will be debilitated since the lungs are unable to perform gas exchange at a satisfactory rate.

One further aspect of alveolar wall destruction is that the airflow between neighboring air sacs, known as collateral ventilation or collateral air flow, is markedly increased as when compared to a healthy lung. While alveolar wall destruction decreases resistance to collateral ventilation, the resulting increased collateral ventilation does not benefit the individual since air is still unable to flow into and out of the lungs. Hence, because this trapped air is rich in CO₂, it is of little or no benefit to the individual.

Chronic bronchitis is characterized by excessive mucus production in the bronchial tree. Usually there is a general increase in bulk (hypertrophy) of the large bronchi and chronic inflammatory changes in the small airways. Excessive amounts of mucus are found in the airways and semisolid plugs of this mucus may occlude some small bronchi. Also, the small airways are usually narrowed and show inflammatory changes.

Currently, although there is no cure for COPD, treatment includes bronchodilator drugs, and lung reduction surgery. The bronchodilator drugs relax and widen the air passages thereby reducing the residual volume and increasing gas flow permitting more oxygen to enter the lungs. Yet, bronchodilator drugs are only effective for a short period of time and require repeated application. Moreover, the bronchodilator drugs are only effective in a certain percentage of the population of those diagnosed with COPD. In some cases, patients suffering from COPD are given supplemental oxygen to assist in breathing. Unfortunately, aside from the impracticalities of needing to maintain and transport a source of oxygen for everyday activities, the oxygen is only partially functional and does not eliminate the effects of the COPD. Moreover, patients requiring a supplemental source of oxygen are usually never able to return to functioning without the oxygen.

Lung volume reduction surgery is a procedure which removes portions of the lung that are over-inflated. The improvement to the patient occurs as a portion of the lung that remains has relatively better elastic recoil which allows for reduced airway obstruction. The reduced lung volume also improves the efficiency of the respiratory muscles. However, lung reduction surgery is an extremely traumatic procedure which involves opening the chest and thoracic cavity to remove a portion of the lung. As such, the procedure involves an extended recovery period. Hence, the long term benefits of this surgery are still being evaluated. In any case, it is thought that lung reduction surgery is sought in those cases of emphysema where only a portion of the lung is emphysematous as opposed to the case where the entire lung is emphysematous. In cases where the lung is only partially emphysematous, removal of a portion of emphysematous lung which was compressing healthier portions of the lung allows the healthier portions to expand, increasing the overall efficiency of the lung. If the entire lung is emphysematous, however, removal of a portion of the lung removes gas exchanging alveolar surfaces, reducing the overall efficiency of the lung. Lung volume reduction surgery is thus not a practical solution for treatment of emphysema where the entire lung is diseased.

Both bronchodilator drugs and lung reduction surgery fail to capitalize on the increased collateral ventilation taking place in the diseased lung. There remains a need for a medical procedure that can alleviate some of the problems caused by COPD. There is also a need for a medical procedure that alleviates some of the problems caused by COPD irrespective of whether a portion of the lung, or the entire lung is emphysematous. The production and maintenance of collateral openings through an airway wall allows air to pass directly out of the lung tissue responsible for gas exchange. These collateral openings serve to decompress hyper inflated lungs and/or facilitate an exchange of oxygen into the blood.

Methods and devices for creating, and maintaining collateral channels are discussed in U.S. patent application Ser. No. 09/633,651, filed on Aug. 7, 2000; U.S. patent application Ser. Nos. 09/947,144, 09/946,706, and 09/947,126 all filed on Sep. 4, 2001; U.S. Provisional Application Nos. 60/317,338 filed on Sep. 4, 2001; 60/334,642 filed on Nov. 29, 2001; 60/367,436 filed on Mar. 20, 2002; 60/374,022 filed on Apr. 19, 2002; 60/387,163 filed on Jun. 7, 2002; and 60/393,629 filed on Jul. 3, 2002 each of which is incorporated by reference herein in its entirety.

Events that may arise when a device is implanted in a surgically-created channel in a lung is that the device can be ejected, filled in with tissue, or otherwise rendered ineffective as the wound heals. It is desirable to provide a device which is capable of providing long-term patency of surgically-created channels in the lung and, in particular, to provide a device which is less susceptible to the above mentioned events.

### BRIEF SUMMARY OF THE INVENTION

This relates to devices and methods for altering gaseous flow in a diseased lung. The conduits described herein maintain the patency of an opening or channel created in the lung tissue. The conduits may comprise a radially expandable center section having a first end and a second end and a passageway extending between the first and second ends. The conduit may further include at least one center-control segment configured to restrict radial expansion of the passageway to a maximum profile. The center-control segment may be designed such that it is curved or slack and when the center section radially expands, the center-control segment tends to straighten. The maximum profile of the center section is reached when the center-control segment becomes substantially straight or taut and hence, no more radial expansion may take place. The center-control segment may be integral with the center section or it may be separately joined to the center section at two or more locations.

The conduit also includes at least one extension member extending from each of the ends of the center section. The extension members are fixed at one end to the center section. The extension members also have a free or movable end such that they may bend about the center section and engage tissue. In particular, the extension members may be outwardly deflected such that opposing extension members sandwich a portion of the lung tissue therebetween. When deployed, opposing extension members may have a V, U, H or other type of shape when viewed from the side. In any event, opposing extension members serve to secure the conduit in the channel of the tissue wall.

The extension members may vary widely in their structure. The extension members may be petal-shaped and they may be arranged around a circumference of the center section. The extension members may be open framed or solid. Additionally, the extension members may be joined or tethered to one another with an extension-control member. The number of extension members connected to the center section may also vary. In one configuration, at least three extension members are attached to each end of the center section of the conduit. However, the invention is not so limited and more or less extension members may be provided. Also, the number of extension members present on one end may be different than the number of extension members present on the other end.

In one variation, the center section comprises a mesh or open-frame structure formed of a plurality of ribs. A center-control segment may be provided which joins adjacent ribs. Also, the center-control segments may join nonadjacent ribs or locations. The center-control member may have various shapes including an arcuate, a semi-circular shape, a circular shape, or other shapes. Additionally, the conduit may comprise at least one ancillary center-control segment to reinforce the primary or first center-control segment. The center-control segments may be identical to one another or they may be different. Also, the center-control segment may be elastic. The center-control segment may also be integral with the center section or it may be a separate component joined thereto.

The center section and portions of the extension members may be coaxially covered with a tissue barrier to prevent tissue ingrowth. The tissue barrier may comprise a material selected from the group consisting of silicone, polyurethane, PET, PTFE, expanded PTFE, and a thin foil metal. Also, the tissue barrier may be located on the exterior or the interior of the center section. The tissue barrier may also be formed in spaces in the side walls of the center section. Additionally, the tissue barrier may cover a portion or all of the extension members such that a distal portion of the extension members remains uncovered. The distal region of the extension members which remains uncovered is susceptible to tissue ingrowth and assists in anchoring the conduit in a channel. In one variation, each and every extension member is partially covered with the tissue barrier.

The conduits described herein may also include a visualization feature about the center section such that the center section may be observed during deployment. The visualization feature may be a stripe surrounding the center section. The visualization feature may be a biocompatible polymer and it may be colored white. In one variation, the visualization feature is shaped like a ring. The visualization feature may also be a visible layer disposed over a portion of the tissue barrier. The visible layer may further be covered by a clear layer of material such as silicone.

A method for deploying a conduit comprises the steps of advancing a delivery device into an airway and deploying the conduit in a channel created in the airway wall. The conduit includes a center section, a plurality of proximal extension members at a proximal end of the center section and a plurality of distal extension members at a distal end of the center section. The method also includes advancing the delivery device through the channel and deploying the extension members of the conduit from the delivery device to engage the tissue. The act of advancing the delivery device at least partially through the channel may comprise: locating the channel with a guide wire; advancing the guide wire through the channel; and advancing the delivery device over the guide wire to advance the delivery device at least partially through the channel.

Also, the step of advancing the delivery device may comprise aligning a visualization feature on the conduit relative to the channel. The visualization feature may be a white ring circumferentially surrounding at least a portion of the center section. Additionally, the act of deploying the extension members of the conduit from the delivery device to engage the tissue may comprise inflating a balloon within the conduit to expand the conduit and bending the extension members about the center section of the conduit such that the extension members engage the tissue wall.

The devices and methods described herein also serve to maintain the patency of a channel surgically created in an airway wall. In particular, the methods and devices prevent closure of the channel such that air may flow through the channel and into the airway. The step of preventing closure of the airway may be performed a number of ways including (1.) impeding the wound healing process of the lung tissue such that the lung tissue cannot heal and the channel remains patent; or (2.) accelerating the wound healing process such that the channel remains patent. Accelerating the wound healing process may be carried out, for example, by increasing the growth of epithelial cells.

The step of preventing closure may comprise inserting a conduit in the channel wherein the conduit includes a passageway for air to flow through.

The step of preventing closure may also be carried out by treating the lung tissue with a bioactive substance. Bioactive substances may be delivered to the channel tissue using various delivery vehicles such as a conduit. The bioactive substance may be disposed on an exterior surface of the conduit such that it interacts with the channel tissue when the conduit is placed at the injury site.

Also, bioactive substances may be delivered to the channel tissue before or after the conduit is positioned in the channel.

Substances which are known to prevent infection may also be used in the present invention. Antibiotics, for example, and other infection-fighting substances can serve to prevent additional wound healing processes which normally commence when an infection or bacteria is present at a wound or injury site.

Conduits for maintaining the patency of a channel created in tissue may comprise a radially expandable center section having a first end and a second end and a passageway extending between the ends. The conduit may further include at least one center-control segment configured to restrict radial expansion of the passageway to a maximum profile. At least one extension member may extend from each of the first and second ends of the center section and each of the extension members may have a fixed end connected to one of the ends of the center section and a movable end such that each of the extension members is capable of being deflected about the fixed end. The conduit further includes a bioactive substance disposed on at least a portion of a surface of the conduit. The bioactive substance may serve to reduce tissue growth such that the conduit remains in the channel and the passageway remains at least partially open. The bioactive substance may be disposed on regions of the surface corresponding to the center section, the extension members, both the center section and extension members, or portions of these features.

Various bioactive substances may be used to prevent the channels from closing. These substances include, for example, infection-fighting substances, wound healing-accelerating substances, and in particular, substances that are known to prevent closure in channels surgically created in the lung airways. Examples of substances include pyrolitic carbon, titanium-nitride-oxide, paclitaxel, fibrinogen, collagen, thrombin, phosphorylcholine, heparin, rapamycin, radioactive 188Re and 32P, silver nitrate, dactinomycin, sirolimus, cell adhesion peptide. However, other substances may be used with the conduits described herein. Also, additional layers of substances may be disposed over the primary bioactive layer. That is to say, more than one bioactive layer or multiple layers of bioactive substances may be deposited on the exterior surface of a conduit device.

The conduit may comprise a mesh formed from a plurality of ribs. Also, the conduit may include a center-control segment which connects at least one rib to an adjacent rib. The center-control segment restricts radial expansion of the conduit to a maximum outer dimension. Additionally, the conduit may comprise a tissue barrier coaxially covering the passageway. The tissue barrier may form an exterior surface upon which the bioactive substance is disposed or the tissue barrier may be integral with or entirely composed of the bioactive substance. The tissue barrier may further cover at least a portion of the extension members or the entire lengths of the extension members.

Another conduit for maintaining the patency of a channel created in tissue comprises a radially expandable center section and extension members as described above. A bioactive substance is disposed on at least a portion of a surface of the conduit. Also, when the conduit is radially expanded it has an overall length and an inner diameter such that a ratio of the overall length to the inner diameter ranges from 1/6 to 2/1. The conduit may also be provided such that this ratio ranges from 1/4 to 1/1 and perhaps, 1/4 to 1/2. A tissue barrier may be disposed on at least a portion of the exterior surface corresponding to the center section. The tissue barrier may be comprised of various materials including but not limited to polymers and elastomers. An example of a material which may be used for the tissue barrier is silicone.

In another variation of the present invention, the conduit includes at least one hold-down member extending from the tips (or another location) of the deflecting members. The hold-down members serve to prevent the conduit from being ejected. The hold-down members desirably include one or more regions which are susceptible to tissue ingrowth or overgrowth. In some embodiments of the present invention, the hold-down members include spaces for tissue to grow into such that it may reconnect with itself, encapsulating the hold-down member and thus preventing ejection of the conduit.

The hold-down member may have a variety of shapes. It may be shaped as, for example, a disk, a “T”, spherical, triangular, a wedge, a ring, looped, hooked, barbed, etc. The hold-down member may also be configured to link one of the deflecting members to an adjacent deflecting member. Also, the hold-down member may extend independently from each deflecting member.

The conduit may comprise at least one visualization feature disposed on a portion of the tissue barrier. The visualization feature may be a stripe circumferentially disposed about at least a portion of the center section or it may be disposed on the extension members or the hold-down members. The visualization feature serves to aid in placement or deployment of the conduit in a target site.

In another variation of the present invention, the conduit includes a braid or mesh at least partially covering the tissue barrier. The braid or mesh is comprised of a plurality of elongated members woven, tied, or otherwise arranged to cover at least a portion of the tissue barrier. The braid or mesh includes spaces between its elongate wire members in which tissue may fill.

In another variation, the conduit includes an exterior porous layer which includes pores, holes or cavities. The exterior covering may also comprise a porous structure. The pores are preferably sized to allow tissue growth therein.

Still another variation of the present invention includes a textured exterior layer. The texture layer is intended to frictionally engage the tissue at the target site such that the likelihood of ejection is reduced. The texture may comprise dimples, dents, etc and is disposed on the surface of the tissue barrier or it may be disposed on the surface of another outer layer which is in a coaxial arrangement with the tissue barrier. The texture may be continuous or segmented. Texture may also be provided on ends or edges of the conduit. Also, the texture may vary in its shape. In one variation, the texture has a saw-tooth pattern. In another variation, the exterior layer has elongated cuts or serrations.

### BRIEF DESCRIPTION OF THE DRAWINGS

FIGS. 1A-1C illustrate various states of the natural airways and the blood-gas interface.

FIG. 1D illustrates a schematic of a lung demonstrating a principle of the invention described herein.

FIG. 2A illustrates a side view of a conduit in an undeployed state.

FIG. 2B illustrates a side view of the conduit of FIG. 2A shown in a deployed shape.

FIG. 2C illustrates a front view of the conduit shown in FIG. 2B.

FIG. 2D is a cylindrical projection of the undeployed conduit shown in FIG. 2A.

FIG. 2E illustrates a side view of another conduit in an undeployed shape.

FIG. 2F illustrates a side view of the conduit of FIG. 2E in a deployed state.

FIG. 2G is a cylindrical projection of the undeployed conduit shown in FIG. 2E.

FIG. 3A illustrates a side view of another conduit having a tissue barrier in a deployed state.

FIG. 3B illustrates a side view of another conduit having a tissue barrier.

FIG. 3C is a front view of the conduit shown in FIG. 3B.

FIG. 3D illustrates a conduit positioned in a channel created in a tissue wall.

FIGS. 3E-3J illustrate various conduits in a deployed state having a tissue barrier and various types of hold-down members.

FIGS. 3K-3N illustrate various conduits in a deployed state having an exterior braid or mesh.

FIG. 3O illustrates a side view of a conduit in a deployed state having an exterior porous layer.

FIG. 3P is a side view of a conduit in a deployed state having a microstructure along its ends.

FIG. 3Q is an enlarged view of a portion of the conduit shown in FIG. 3P.

FIG. 3R illustrates a side view of a conduit in a deployed state having an exterior layer with elongated cuts.

FIG. 3S is a cross sectional view of the conduit shown in FIG. 3B taken along line A-A.

FIGS. 4A-4C illustrate a method for deploying a conduit.

FIGS. 5A-5B illustrate a method for deploying a conduit at an angle.

### DETAILED DESCRIPTION OF THE INVENTION

Described herein are devices and methods for improving the gaseous exchange in the lung. In particular, a conduit is described that serves to maintain collateral openings or channels surgically created through an airway wall so that air is able to pass directly out of the lung tissue and into the airways. This facilitates exchange of oxygen into the blood and decompresses hyper inflated lungs.

By “channel” it is meant to include, but not be limited to, any opening, hole, slit, channel or passage created in the airway wall. The channel may be created in tissue having a discrete wall thickness and the channel may extend all the way through the wall. Also, a channel may extend through lung tissue which does not have well defined boundaries such as, for example, parenchymal tissue.

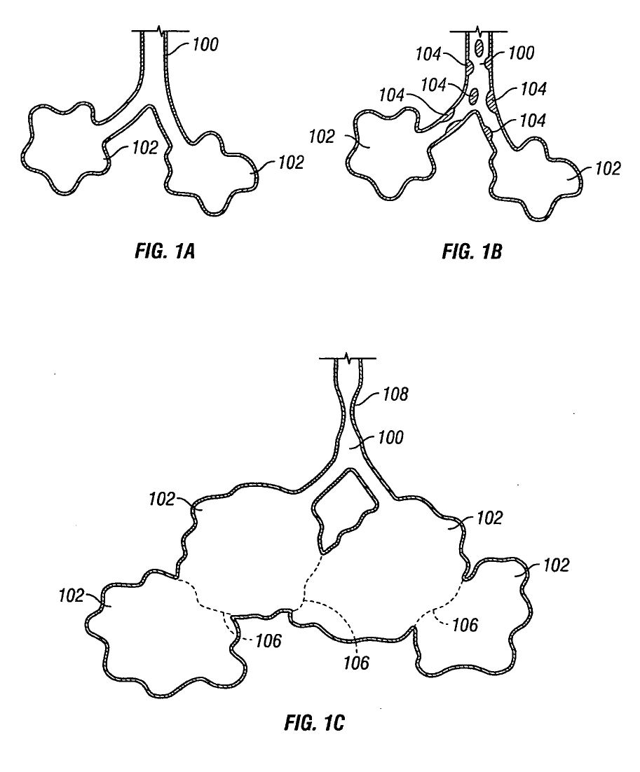

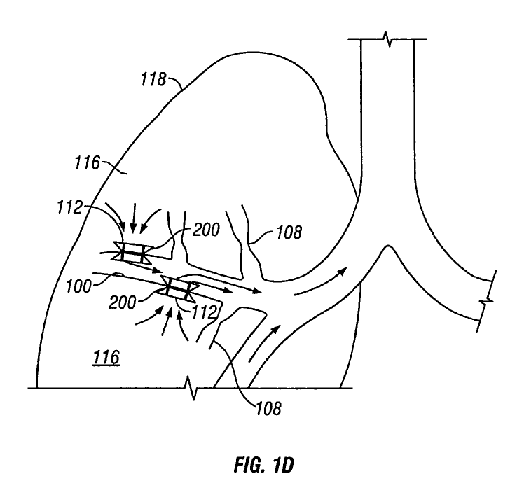

As stated above, the conduits described herein may improve airflow through an airway in the lung. Simplified illustrations of various states of a natural airway and a blood gas interface found at a distal end of those airways are provided in FIGS. 1A-1C. FIG. 1A shows a natural airway 100 which eventually branches to a blood gas interface 102. FIG. 1B illustrates an airway 100 and blood gas interface 102 in an individual having COPD. The obstructions 104 impair the passage of gas between the airways 100 and the interface 102. FIG. 1C illustrates a portion of an emphysematous lung where the blood gas interface 102 expands due to the loss of the interface walls 106 which have deteriorated due to a bio-chemical breakdown of the walls 106. Also depicted is a constriction 108 of the airway 100. It is generally understood that there is usually a combination of the phenomena depicted in FIGS. 1A-1C. Often, the states of the lung depicted in FIGS. 1B and 1C may be found in the same lung.

FIG. 1D schematically illustrates airflow in a lung 118 when conduits 200 are placed in collateral channels 112. As shown, collateral channels 112 (located in an airway wall) place lung tissue 116 in fluid communication with airways 100 allowing air to pass directly out of the airways 100 whereas constricted airways 108 may ordinarily prevent air from exiting the lung tissue 116. While the invention is not limited to the number of collateral channels which may be created, it is to be understood that 1 or 2 channels may be placed per lobe of the lung and perhaps, 2-12 channels per individual patient. However, as stated above, the invention includes the creation of any number of collateral channels in the lung. This number may vary on a case by case basis. For instance, in some cases in an emphysematous lung, it may be desirable to place 3 or more collateral channels in one or more lobes of the lung.

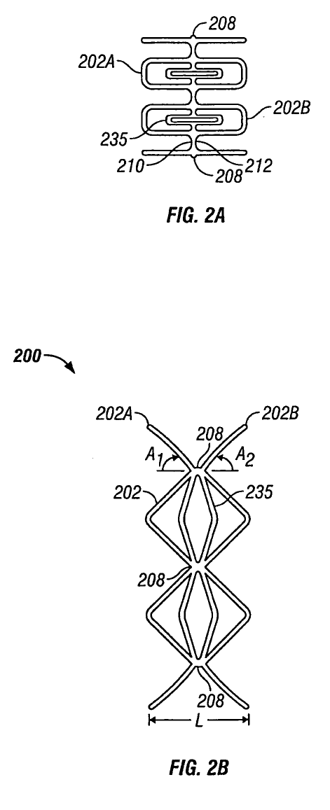

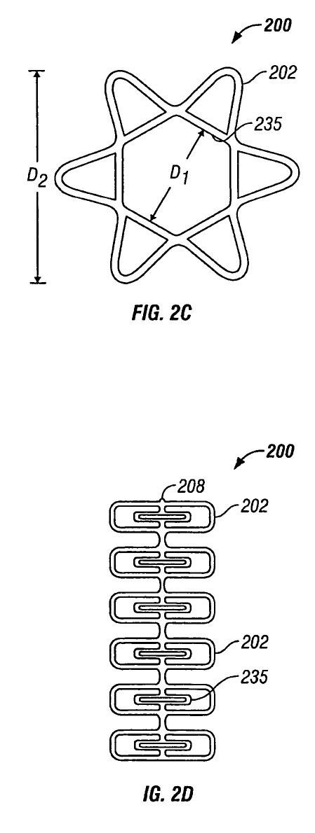

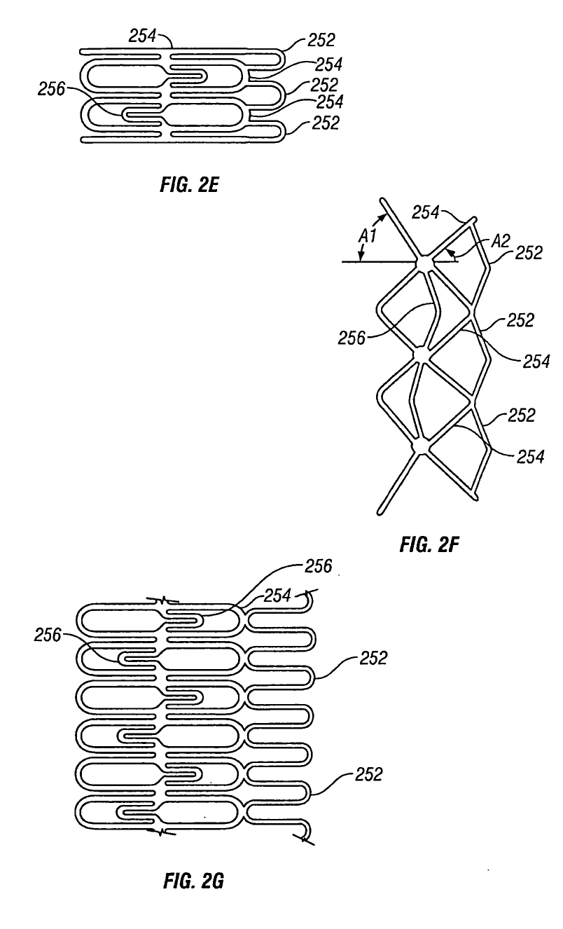

As shown in FIGS. 2A-2G, the conduits described herein generally include a center section 208 and at least one extension member (or finger) 202A, 202B extending from each end of the center section. The extension members, as will be discussed in more detail below, are capable of deflecting or outwardly bending to secure the conduit in an opening created in an airway wall thereby maintaining the patency of the opening. The extension members may deflect such that opposing extension members may form a V, U or other type of shape when viewed from the side.

Additionally, the conduits shown in FIGS. 2A-2G include a center-control segment 235 which restricts or limits radial expansion of the center section. The center-control segments are adapted to straighten as the center section is radially expanded. Once the center-control segments become straight or nearly straight, radial expansion of the conduit is prevented. In this manner, the radial expansion of the conduit may be self controlled.

The conduits described herein may have various states (configurations or profiles) including but not limited to (1.) an undeployed state and (2.) a deployed state.

The undeployed state is the configuration of the conduit when it is not secured in an opening in an airway wall and, in particular, when its extension members (or fingers) are not outwardly deflected to engage the airway wall. FIG. 2A is a side view of a conduit 200 in an undeployed state. As shown in this figure, extension members 202A, 202B extend straight from the ends 210, 212 respectively of center section 208. The extension members shown in this example are parallel. However, the invention is not so limited and the extension members need not be parallel.

The deployed state is the configuration of the conduit when it is secured in a channel created in an airway wall and, in particular, when its extension members are outwardly bent to engage the airway wall such that the conduit is fixed in the opening. An example of a conduit in its deployed configuration is shown in FIGS. 2B and 2C. FIG. 2B is a side view of a conduit in its deployed state and FIG. 2C shows a front view of the conduit of FIG. 2B.

As shown in FIGS. 2A-2D, the conduit includes a center section 208 having a short passageway. This center section may be a tubular-shaped open-frame (or mesh) structure having a plurality of ribs. Also, as explained in more detail below, the center section may be a sheet of material.

The axial length of the center section or passageway may be relatively short. In FIGS. 2A-2D, the passageway's length is about equal to the width of a wire segment or rib. Here, the center section serves as a bridge or junction for the extension members and it is not required to be long. The axial length of the passageway may therefore be less than 1 mm and even approach 0 mm. In one example, the length of the center section is less than twice the square root of a cross sectional area of the center section. However, the center section may also have passageways which have lengths greater than 1 mm.

The overall length (L) of the conduit may be distinguished from the length of the center section because the overall length includes the lengths of the extension members. Further, the overall length (L) is dependent on which state the conduit is in. The overall length of the conduit will typically be shorter when it is in a deployed state as shown in FIG. 2B than when it is in an undeployed state as shown in FIG. 2A. The overall length (L) for a deployed conduit may be less than 6 mm and perhaps, between 1 and 20 mm.

FIG. 2C shows a front view of the conduit 200 shown in FIG. 2B. FIG. 2C shows the passageway having a hexagonal (or circular) cross section. The cross-section, however, is not so limited. The cross section may be circular, oval, rectangular, elliptical, or any other multi-faceted or curved shape. The inner diameter (D₁) of the center section, when deployed, may range from 1 to 10 mm and perhaps, from 2 to 5 mm. Moreover, in some variations, the cross-sectional area of the passageway, when deployed, may be between 0.2 mm² to 300 mm² and perhaps between 3 mm² and 20 mm².

The diameter of the center section, when deployed, thus may be significantly larger than the passageway's axial length (e.g., a 3 mm diameter and an axial length of less than 1 mm). This ratio of the center section length to diameter (D₁) may range from about 0:10 to 10:1, 0.1:6 to 2:1 and perhaps from 1:2 to 1:1.

The diameter of the center section, when deployed, may also be nearly equal to the overall length (L) of the conduit 200. This overall length (L) to diameter (D₁) ratio may range from 1:10 to 10:1, 1:6 to 2:1, and perhaps from 1:4 to 1:1. However, the invention is not limited to any particular dimensions or ratio. Rather, the conduit should have a center section such that it can maintain the patency of a collateral channel in an airway wall. The dimensions of the center section (and the conduit as a whole) may be chosen based on the tissue dimensions. When the channel is long in its axial length, for example, the length of the center section may likewise be long or identical to the channel's length.

As mentioned above, extending from the ends of the center section 208 are extension members 202A, 202B which, when the conduit is deployed, form angles A1, A2 with a central axis of the passageway. The extension members may bend or deflect about the center section or they may be adapted to bend or deflect at a point along their lengths. When viewed from the side such as in FIG. 2B, opposing extension members may have a V, U, or other shape. The extension members 202A, 202B may thus outwardly rotate until they sandwich tissue (not shown) between opposing extension members.

The angles A1, A2 may vary and may range from, for example, 30 to 150 degrees, 45 to 135 degrees and perhaps from 30 to 90 degrees. Opposing extension members may thus form angles A1 and A2 of less than 90 degrees when the conduit is deployed in a channel. For example, angles A1 and A2 may range from 30 to 60 degrees when the conduit is deployed.

The conduits of the present invention are effective and may maintain a surgically created opening despite not substantially sandwiching tissue between opposing extension members as described above. Additionally, it is not necessary for the conduits of the present invention to prevent air from flowing along the exterior of the conduit. That is, air may move into (and through) spaces between the exterior of the conduit and the interior wall of the tissue channel. Thus, fluidly sealing the edges of the conduit to prevent side flow or leakage around the conduit is not crucial for the conduits to be effective. However, the conduits of the present invention are not so limited and may reduce or eliminate side flow by, for example, increasing the angles A1 and A2 and adding sealant around the exterior of the conduit.

The angle A1 may be different than angle A2. Accordingly, the conduit may include proximal extension members which are parallel (or not parallel) to the distal extension members. Additionally, the angle corresponding to each proximal extension member may be different or identical to that of another proximal extension member. Likewise, the angle corresponding to each distal extension member may be different or identical to that of another distal extension member.

The extension members may have a length between 1 and 20 mm and perhaps, between 2 and 6 mm. Also, with reference to FIG. 2C, the outer diameter (D₂) of a circle formed by the free ends of the extension members may range from 2 to 20 and perhaps, 3 to 10 mm. However, the invention is not limited to the dimensions disclosed above. Furthermore, the length of the distal extension members may be different than the length of the proximal extension members. The length of the distal extension members may be, for example, longer than that of the proximal extension members. Also, the lengths of each proximal extension member may be different or identical to that of the other proximal extension members. Likewise, the lengths of each distal extension member may be different or identical to that of the other distal extension members.

The number of extension members on each end of the center section may also vary. The number of extension members on each end may range from 2-10 and perhaps, 3-6. Also, the number of proximal extension members may differ from the number of distal extension members for a particular conduit. Moreover, the extension members may be symmetrical or non-symmetrical about the center section. The proximal and distal extension members may also be arranged in an in-line pattern or an alternating pattern. The extension members or the center section may also contain barbs or other similar configurations to increase adhesion between the conduit and the tissue. The extension members may also have openings to permit tissue ingrowth for improved retention.

The shape of the extension members may also vary. They may be open-framed and somewhat petal-shaped as shown in FIGS. 2A-2D. In these figures, the extension members 202A, 202B comprise wire segments or ribs that define openings or spaces between the members. However, the invention is not so limited and the extension members may have other shapes. The extension members may, for example, be solid or they may be filled.

In another variation the conduit is constructed to have a delivery state. The delivery state is the configuration of the conduit when it is being delivered through a working channel of a bronchoscope, endoscope, airway or other delivery tool. The maximum outer diameter of the conduit in its delivery state must therefore be such that it may fit within the delivery tool, instrument, or airway.

In one variation, the conduit is radially expandable such that it may be delivered in a smaller working channel of a scope while maximizing the diameter to which the conduit may expand upon deployment. For example, sizing a conduit for insertion into a bronchoscope having a 2 mm or larger working channel may be desirable. Upon deployment, the conduit may be expanded to have an increased internal diameter (e.g., 3 mm.) However, the invention is not limited to such dimensions. It is contemplated that the conduits 200 may have center sections that are expanded into a larger profile from a reduced profile, or, the center sections may be restrained in a reduced profile, and upon release of the restraint, return to an expanded profile.

Additionally, the conduit need not have a smaller delivery state. In variations where the center section is not able to assume a second smaller delivery profile, a maximum diameter of the first or deployed profile will be sufficiently small such that the conduit may be placed and advanced within an airway or a working channel of a bronchoscope or endoscope. Also, in cases where the conduit is self-expanding, the deployed shape may be identical to the shape of the conduit when the conduit is at rest or when it is completely unrestrained.

The conduit 200 shown in FIGS. 2A-2D also includes diametric-control segments, tethers, or leashes 235 to control and limit the expansion of the center section 208 when deployed. This center-control segment 235 typically is shaped such that when the conduit radially expands, the center-control segment bends until it is substantially straight or no longer slack.

By ‘slack’ we mean, for example, that the control segment(s) is not in a state of tension such that it opposes further expansion of the conduit or a section thereof. After the conduit is fully deployed/expanded, the segment(s) may or may not remain in a state of tension.

Such a center-control segment 235 may be circular or annular shaped. However, its shape may vary widely and it may have, for example, an arcuate, semi-circular, V, or other type of shape which limits the expansion of the conduit.

Typically, one end of the center-control segment is attached or joined to the center section at one location (e.g., a first rib) and the other end of the center-control segment is connected to the center section at a second location (e.g., a rib adjacent or opposite to the first rib). However, the center-control segments may have other constructs. For example, the center-control segments may connect adjacent or non-adjacent center section members. Further, each center-control segment may connect one or more ribs together. The center-control segments may further be doubled up or reinforced with ancillary control segments to provide added control over the expansion of the center section. The ancillary control segments may be different or identical to the primary control segments.

FIG. 2B illustrates the conduit 200 in its deployed configuration. As discussed above, the center-control segments 235 may bend or otherwise deform until they maximize their length (i.e., become substantially straight) such as the center-control segments 235 shown in FIG. 2B. However, as discussed above, the invention is not so limited and other types of center-control segments may be employed.

As shown in FIGS. 2E-2G, control segments 252 may also be used to join and limit the expansion of the extension members 254 or the control segments may be placed elsewhere on the conduit to limit movement of certain features to a maximum dimension. By controlling the length of the control segments, the shape of the deployed conduit may be controlled. In the conduit shown in FIGS. 2E-2G, the conduit includes both center-control segments 256 and distal control segments 252. The center-control segments are arcuate shaped and join adjacent rib sections of the center section and the distal-control segments are arcuate and join adjacent distal extension members.

FIG. 2F illustrates the conduit in a deployed configuration and shows the various control members straightening as the extension members and center section deploy. The proximal extension members, however, are not restricted by a control member and consequently may be deflected to a greater degree than the distal extension members. Accordingly, a conduit having control members connecting, for example, regions of the center section and having additional control segments connecting extension members, may precisely limit the maximum profile of a conduit when it is deployed. This is desirable where overexpansion of the conduit is hazardous.

This also serves to control the deployed shape of the conduit by, for instance, forcing angle A1 to differ from angle A2. Using control segments in this manner can provide for cone-shaped conduits if the various types of control-segments have different lengths. For example, providing longer proximal-control segments than distal-control segments can make angle A1 larger than angle A2. Additionally, cylindrical-shaped conduits may be provided if the center-control segments and the extension-control segments are sized similarly such that angle A1 equals angle A2. Again, the control segments straighten as the conduit expands and the conduit is thus prevented from expanding past a predetermined amount.

Furthermore, a variation of the conduit may have extension control members of varying lengths so that upon expansion the conduit takes a shape other than a tubular shape (e.g., oval, rectangular, square, etc.)

The control segments, as with other components of the conduit, may be added or mounted to the center section or alternatively, they may be integral with the center section. That is, the control segments may be part of the conduit rather than separately joined to the conduit with adhesives or welding, for example. The control segments may also be mounted exteriorly or interiorly to the members to be linked.

Additionally, sections of the conduit may be removed to allow areas of the conduit to deform more readily. These weakened areas provide another approach to control the final shape of the deployed conduit. Details for creating and utilizing weakened sections to control the final shape of the deployed conduit may be found in U.S. Pat. No. 09/947,144 filed on Sep. 4, 2001.

The conduit described herein may be manufactured by a variety of manufacturing processes including but not limited to laser cutting, chemical etching, punching, stamping, etc. For example, the conduit may be formed from a tube that is slit to form extension members and a center section between the members. One variation of the conduit may be constructed from a metal tube, such as stainless steel, 316L stainless steel, titanium, titanium alloy, nitinol, MP35N (a nickel-cobalt-chromium-molybdenum alloy), etc. Also, the conduit may be formed from a rigid or elastomeric material that is formable into the configurations described herein. Also, the conduit may be formed from a cylinder with the passageway being formed through the conduit. The conduit may also be formed from a sheet of material in which a specific pattern is cut. The cut sheet may then be rolled and formed into a tube. The materials used for the conduit can be those described above.

Additionally, the conduits described herein may be comprised of a shape memory alloy, a super-elastic alloy (e.g., a NiTi alloy), a shape memory polymer, a polymeric material, an implantable material, a material with rigid properties, a material with elastomeric properties, or a combination thereof. The conduit may be constructed to have a natural self-assuming deployed configuration, but is restrained in a pre-deployed configuration. As such, removal of the restraints causes the conduit to assume the deployed configuration. A conduit of this type could be, but is not limited to being, comprised from a shape memory alloy. It is also contemplated that the conduit could comprise a shape memory alloy such that, upon reaching a particular temperature (e.g., 98.5° F.), it assumes a deployed configuration.

Also, the conduit described herein may be formed of a plastically deformable material such that the conduit is expanded and plastically deforms into a deployed configuration. The conduit may be expanded into its expanded state by a variety of devices such as, for example, a balloon catheter.

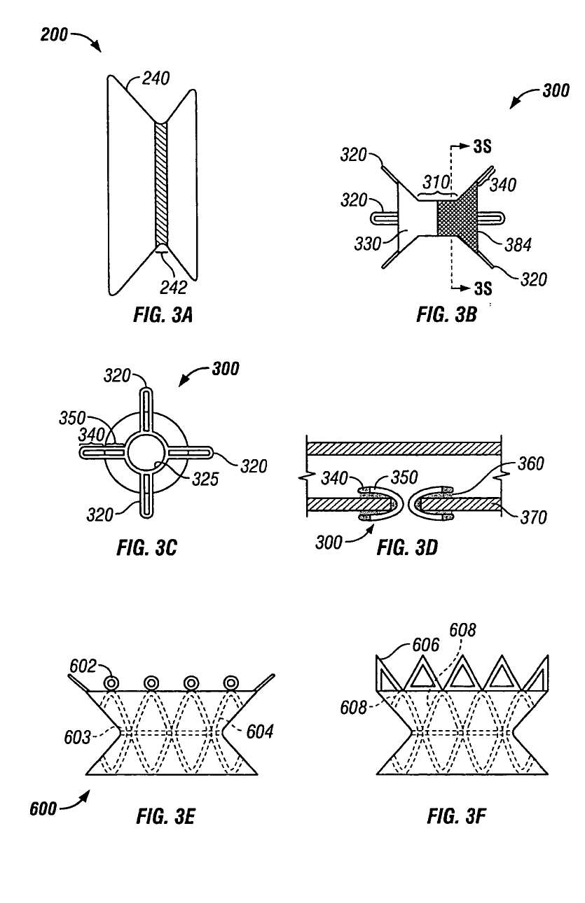

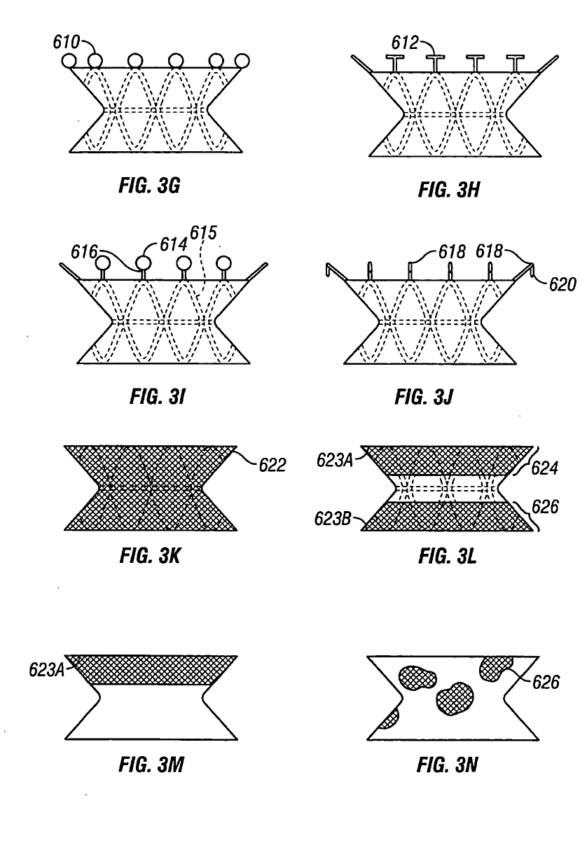

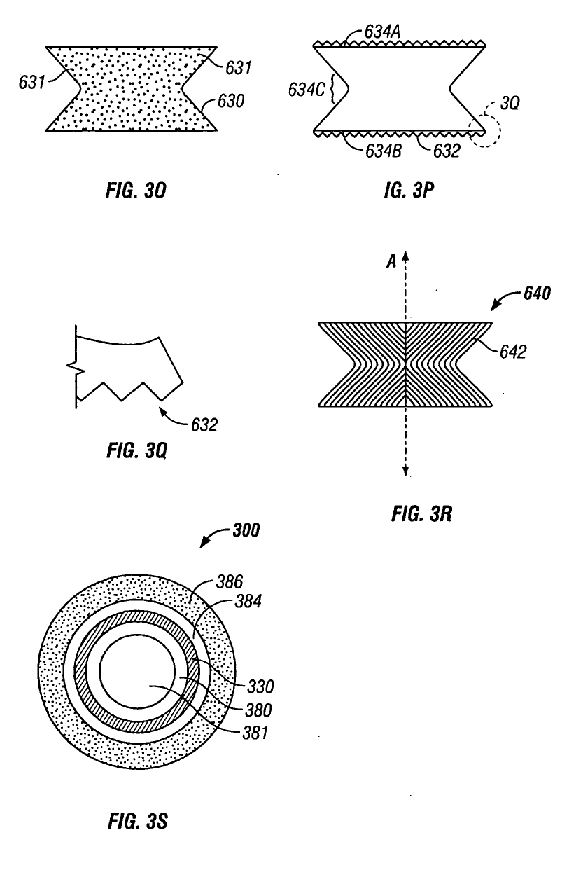

FIG. 3A illustrates another variation of a conduit 200 having a tissue barrier 240. The tissue barrier 240 prevents tissue ingrowth from occluding the collateral channel or passage of the conduit 200. The tissue barrier 240 may coaxially cover the center section from one end to the other or it may only cover one or more regions of the conduit 200. The tissue barrier may completely or partially cover the conduit. The tissue barrier 240 may be located about an exterior of the conduit's surface, about an interior of the conduit's surface, or the tissue barrier 240 may be located within openings in the wall of the conduit's surface. Furthermore, in some variations of the invention, the center section 208 itself may provide an effective barrier to tissue ingrowth. The tissue barrier, of course, should not cover or block the entrance and exit of the passageway such that air is prevented from passing through the conduit's passageway. However, in some constructs, the tissue barrier may partially block the entrance or exit of the passageway so long as air may continue to pass through the conduit's passageway.

The tissue barrier may be formed from a material, or coating that is a polymer or an elastomer such as, for example, silicone, polyurethane, PET, PTFE, or expanded PTFE. Moreover, other biocompatible materials will work, such as a thin foil of metal, etc. The coatings may be applied, for example, by either dip coating, molding, spin-coating, transfer molding or liquid injection molding. Or, the tissue barrier may be a tube of a material and the tube is placed either over and/or within the conduit. The tissue barrier may then be bonded, crimped, heated, melted, shrink fitted to the conduit. The tissue barrier may also be tied to the conduit with a filament of, for example, a suture material. The tissue barrier may also be placed on the conduit by either solvent swelling applications or by an extrusion process. Also, a tissue barrier may be applied by either wrapping a sheet of material about the conduit, or by placing a tube of the material about the conduit and securing the tube to the conduit. Likewise, a tissue barrier may be secured on the interior of the conduit by positioning a sheet or tube of material on the inside of the center section and securing the material therein.

FIGS. 3B and 3C respectively illustrate a side view and a front view of another conduit 300 having a partial tissue barrier coating. The conduit 300 includes a center section 310, a plurality of extension members 320, and a partial tissue barrier 330. The conduit 300 is thus different than that shown in FIG. 3A in that the center section is longer and that the tissue barrier 330 only partially covers the extension members 320. In particular, the center section 310 shown in FIGS. 3B-3C is cylindrical or tubular-shaped. This shape may be advantageous when a relatively longer passageway is desired. Also, it is to be understood that the overall (or three dimensional) shape of the center section, when deployed, is not limited to the shape shown here. Rather, it may have various shapes such as, for example, rectangular, tubular, conical, hour-glass, hemi-toroidal, etc.

Additionally, the tissue barrier 330 covers only a proximal region 350 of the extension members and leaves a distal region 340 of the extension members uncovered. The distal region 340 of the extension members 320 is shown as being open-framed. However, the invention is not so limited. The distal region of the extension members may be solid and it may include indentations, grooves, and recesses for tissue ingrowth. Also, the extension members may include small holes for tissue ingrowth. For example, the distal region of the extension members may have a dense array of small holes. In any event, the conduits described herein may include at least one region or surface which is susceptible to tissue ingrowth or is otherwise adherent to the tissue. Accordingly, tissue ingrowth at the distal region 340 of the extension members is facilitated while tissue growth into the passageway 325 is thwarted.

As shown in FIG. 3D, tissue growth 360 into the uncovered region 340 further secures the extension members to the tissue wall 370. The distal region of the extension members may also include tissue growth substances such as epithelial growth factors or agents to encourage tissue ingrowth. Accordingly, conduit 300 may be configured to engage the tissue wall 370 as well as to allow tissue to grow into predetermined regions of the conduit.

FIGS. 3E to 3J show various conduits in a deployed state each of which has one or more hold-down members. The hold-down members serve to prevent ejection of the conduit from an implantation site such as a surgically created channel in an airway. The hold-down members generally include an aperture or other structure which is susceptible to tissue ingrowth or encapsulation at the injury site. The tissue grows into (or around) the hold-down members securing the conduit in place. In some instances, the tissue can grow through an opening in the hold-down member and reconnect with itself thereby locking the conduit in place.

The hold-down members may have various shapes. FIG. 3E shows a conduit 600 having ring-shaped hold-down members 602 extending from the tips of deflectable extension members 604. The extension members are shown hidden behind a tissue barrier layer which is in coaxial arrangement with the conduit's center section 603 and extension members 604. The tissue barrier may be a polymer coating such as, e.g., a silicone coating.

The rings 602 shown in FIG. 3E are circular and symmetrical. However, the rings may be otherwise shaped. The rings may be oblong or elongated, square, triangular, etc. Additionally, the rings 602 are shown disposed on only one end of the conduit but the invention is not so limited. The hold-down members may be disposed on the distal end, proximal end, both ends, or intermediate of the ends of the conduit. Also, the number of hold-down members present need not equal the number of extension members. There may be, for example, more or less hold-down members than deflectable extension members.

FIG. 3F shows another conduit having hold-down members 606. The hold-down members 606 shown in FIG. 3F are triangular and connect the tips of adjacent extension members 608. While the hold-down members are shown in this figure as triangular, another shape of wire segment may be used to link one extension member with an adjacent extension member so long as the link forms an opening or space for tissue ingrowth.

The hold-down members may also be solid such as the spheres shown in FIG. 3G. Tissue grows around the spheres 610 to secure the conduit in a channel. The diameter of these rounded hold-down members may range from 0.15 to 3 mm and perhaps 0.2 to 1 mm. However, the shapes of the hold-down members may vary and they are not intended to be limited to only the examples provided herein.

FIGS. 3H and 3I show another conduit having hold-down members. The hold-down members serve the same purpose as described above. FIG. 3H shows hold-down members 612 having a T-shape. Of course, the hold-down members may have the shape of other letters, symbols and things such as, for example, a disk. Also, the hold-down members may have the shape of a hook or open-ended loop. FIG. 3I shows disk-shaped hold-down members 614 mounted to the tips of the extension members 615 with a link member 616. The hold-down members 614 of FIG. 3I thus have a similar shape to that of a lollipop.

FIG. 3J shows another conduit having hold-down members 618. The hold-down members 618 have prongs or barbs 620. The barbs are configured to penetrate tissue to further secure the conduit in place. Also, the barbs may be combined with any of the hold-down members described herein unless features mutually exclude such a combination.

While the hold-down members are desirably extensions of (or mounted to) the tips of the deflectable extension members, the hold-down members may be placed anywhere on the conduit's exterior. This may be accomplished by forming the hold-down members with the conduit frame structure and coaxially coating the exterior of the conduit as described in this disclosure. After the coating is formed on the frame structure, the material covering the hold-down members may be cut away thereby exposing the hold-down members. Also, the coating may be controlled such that the hold-down members are not coated. For example, the hold-down members may be covered with a temporary shield while the conduit is spray- or dip-coated with a polymer. Still other techniques for fabricating the conduit with hold-down members may be employed as is known to those of ordinary skill in the art.

The hold-down members may be comprised of metal, plastic, alloys or combinations thereof. The hold-down members may be made of the same material as the frame or body of the conduit. Also, the hold-down members may be formed from the material coating the frame. That is, the coating may be applied to form the hold-down feature or it may be applied as discussed above and then modified to form a loop or other hold-down feature in accordance with the present invention. For example, one hold-down member may be formed of a silicone loop or ring extending from a deflectable or extension member. The silicone loop may be integrally joined with the silicone coating which covers the frame of the conduit.

Also, the hold-down members may have similar dimension and flexibility as the frame members. For example, a thin sheet of metal may be laser cut into a frame having a center section, extension members, and hold-down members. The conduit may then be coated as described above.

FIGS. 3K-3M each depicts a conduit having a wire mesh or braid coaxially surrounding the tissue barrier. FIG. 3K shows a mesh 622 coaxially surrounding the tissue barrier and FIG. 3L shows mesh portions 623A, 623B surrounding only a first portion 624 and second portion 626 of the tissue barrier corresponding to the first set of extension members and second set of extension members respectively. FIG. 3M shows an asymmetrical configuration having a braid 623A surrounding only a first portion of the conduit. FIG. 3N illustrates still another conduit having braid patches 626 covering various portions of the conduit.

The braids are exterior to the surface of the tissue barrier and are used to promote tissue ingrowth to secure the conduit in place. The braid may be placed directly upon the tissue barrier and bonded directly to the tissue barrier in at least one contact location using an adhesive. There may be multiple contact locations distributed evenly or unevenly. The contact locations may be bonded with an adhesive.

The mesh or braid comprises a number of elongated members arranged, tied, or woven together to form the finished exterior cover. The elongate members may be wires having a circular or square cross section or the elongate members may be ribbon-like. The braid may have a single size of wire or ribbon but the braid need not be so limited. Multiple sizes of wires or ribbons may be used as desired.

Additionally, the braid may have a single pitch, an angle of a constituent ribbon measured against the axis of the braid, or it may have a pitch which varies along the axis of the braid.

The elongated members may be made of metals such as steel; they may comprise superelastic alloys; or they may be polymeric. Preferred super-elastic alloys include the class of titanium/nickel materials known as nitinol-alloys. These materials are discussed, amongst other places, in U.S. Pat. Nos. 3,174,851 to Buehler et al., 3,351,463 to Rozner et al., and 3,753,700 to Harrison et al.

Metallic ribbons that are suitable for use in this invention are desirably between 0.25 mil and 3.5 mil in thickness and 2.5 mil and 12.0 mil in width. However, other sizes may be used so long as the conduit may be properly deployed as described herein. Also, by the term “ribbon”, we intend to include elongated shapes, the cross-section of which are not square or round and may typically be rectangular, oval or semi-oval. They should, but are not required to, have an aspect ratio of at least 0.5 (thickness/width). In any event, for super-elastic alloys, particularly nitinol, the thickness and width may be somewhat finer, e.g., down to 0.25 mil and 1.0 mil, respectively. Examples of ribbon sizes are 1 mil×3 mil, 1 mil×4 mil, 2 mil×6 mil, and 2 mil×8 mil.

The ribbons making up the braid may also contain a minor amount of non-super-elastic materials. Fibrous materials (both synthetic and natural) may also be used. Preferred, because of cost, strength, and ready availability are stainless steels (SS304, SS306, SS316, etc.) and tungsten alloys. Also, more malleable metals and alloys, e.g., gold, platinum, palladium, rhodium, etc. may be used. A platinum alloy with a few percent of tungsten may also provide radio-opacity.

The braid or mesh is made of an implantable, perhaps flat, material wrapped around the conduit. Suitable non-metallic materials include polypropylene, nylon, PTFE or other suture materials or other implantable polymer materials. Other materials which may find use in the present invention include those made of polyaramids (e.g., KEVLAR) and carbon fibers. Additionally, the conduit may include an open cell foam covering. For example, natural and synthetic sponges may be wrapped around the conduit and cut to length. The open cell foam materials provide spaces for tissue to grow into and reconnect with itself, securing the conduit in place.

The braids utilized in this invention may be made using commercially available tubular braiding machines. Whenever the term “braid” is used herein, we mean constructions in which the ribbons making up the construction are woven in an in-and-out fashion as they cross to form a covering of the tissue barrier. The braids may be made up of a suitable number of ribbons, typically six or more. Ease of production on a commercial braider typically results in braids having eight or sixteen ribbons.

Also, a braided sheet of interwoven filaments or ribbons may be formed. The sheet can be rolled into a tubular structure and fitted onto a conduit. The braided tubular structure is cut to length and then bonded to the conduit. Still other techniques to form and secure the braid onto the conduit may be employed in accordance with the present invention.

The braid may also be rough to the touch if not covered or further processed. Procedures such as rolling, sanding, or grinding may be used to smooth the surface of the braid if so desired.

Again, the braid or mesh may be formed of various elongate members including wires having a circular cross section as well as ribbons having various cross sections which are not square or circular. The braid or mesh is coaxially disposed over the tissue barrier of the conduit such that tissue may grow into openings or cavities formed between the elongate members. Tissue also may grow into the space between the braid and the tissue barrier. Tissue ingrowth helps to secure the conduit in place preventing ejection.

FIG. 3O shows a configuration of a conduit which includes a porous exterior layer 630. The porous exterior layer includes holes, microholes, pores or cavities which provide a roughened or frictional surface for tissue to grip and grow into when the conduit is deployed in an injury site such as a channel created through an airway wall. The porous layer 630 is exterior to the tissue barrier such that tissue growing into the pores 631 is not able to penetrate the tissue barrier. Of course, the exterior layer 630 does not cover the ends of the conduit such that airflow through the conduit's passageway is prevented.

The exterior layer may be made from a number of substances including polymers. An open cell foam material may be suitable for example. Natural and synthetic sponges may be used. Also, the thickness of the exterior layer should be in the range of 0.01-1 mm and perhaps from 0.05-2 mm.

FIGS. 3P and 3Q depict another conduit in a deployed state having a microstructure 632 protruding from ends 634A, 634B of the conduit. In particular, as shown in FIG. 3Q, the microstructure 632 has a sawtooth shape. Also, while the structure is shown at the ends of the first and second portions of the conduit the microstructure may occupy other areas of the tissue barrier such as, e.g., the center region 634C. These structures may be created by a number of techniques including, for example, molding, sanding, cutting, or roughening selected portions of the tissue barrier. Structures may also be created in the tissue barrier using micromachining and more traditional machining techniques.

FIG. 3R shows a conduit 640 having elongated cuts or projections 642 in its outer surface. The cuts serve to engage tissue and provide elongated regions for tissue ingrowth. Though the cuts 642 are shown running parallel to the passageway, they need not be so aligned. The cuts may run perpendicular to the axis of the passageway A. The cuts may also run at another angle to the axis A of the conduit. Also, the cuts 642 (as well as the other textures and microstructures described herein) may be intermittently disposed on the conduit. Thus the textures may be continuous and uniform or they may be intermittent. Also, one or more types of texture, exterior layers, and hold-down members may be combined to form one conduit. To reiterate, various hold-down members and/or exterior layers may be provided to prevent the conduit from being ejected when deployed in a channel surgically created in an airway of a lung.

The conduits may also include a visualization feature or marker to increase its visibility during a medical procedure. Referring again to FIG. 3A, a conduit is shown having a visualization ring/marker 242. The marker 242 is visually apparent during a procedure. The marker is observed as the conduit is placed in a collateral channel and, when the marker is even with the opening of the channel, the conduit may be deployed. In this manner, the visualization feature facilitates alignment and deployment of the conduits into collateral channels.

The visualization ring or mark may be a biocompatible polymer and have a color such as white. Also, the visualization feature may protrude from the center section or it may be an indentation(s). The visualization mark may also be a ring, groove or any other physical feature on the conduit. Moreover, the visualization feature may be continuous or comprise discrete segments (e.g., dots or line segments).

The visualization feature may be made using a number of techniques. In one example, the mark is a ring formed of silicone and is white. The polymeric ring may be spun onto the tissue barrier. For example, a clear silicone barrier may be coated onto the conduit such that it coaxially covers the extension members and the center section as shown in FIG. 3A. Next, a thin ring of white material such as a metal oxide suspended in clear silicone may be spun onto the silicone coating. Finally, another coating of clear silicone may be applied to coat the white layer. The conduit thus may include upwards of 1-3 layers including a tissue barrier, a visualization mark layer, and a clear outer covering.

The shape of the visualization mark is not limited to a thin ring. The visualization mark may be large, for example, and cover an entire half of the conduit as shown in FIG. 3B. The visualization mark may, for example, be a white coating disposed on the proximal or distal half of the conduit. The visualization mark thus may extend from an end of the extension members to the center section of the conduit. As explained in more detail below, when such a device is deposited into a channel created in lung tissue, the physician may observe when one-half of the conduit extends into the channel. This allows the physician to properly actuate or deploy the conduit to secure the conduit in the tissue wall.

The visualization member described above is visually apparent to a physician using various instruments such as, for example, an endoscope. The visualization feature, however, may also be made of other vision-enhancing materials such as radio-opaque metals used in x-ray detection. It is also contemplated that other elements of the conduit can include visualization features such as but not limited to the extension members, tissue barrier, control segments, hold-down members, etc. Of course when the control segments, extension members, hold-down members, meshes, braids, surface textures and other features of the conduit are visually apparent during a procedure, they can assist in, amongst other things, visualizing the device during a procedure.

The conduits may also include a one-way valve. The valve may be positioned such that it permits expiration of gas from lung tissue but prevents gas from entering the tissue. The valve may be placed anywhere within the passageway of the conduit. The valve may also be used as bacterial in-flow protection for the lungs. The valve may also be used in conjunction with a tissue barrier and the tissue barrier may be disposed coaxially about the conduit. Various types of one way valves may be used as is known to those of skill in the art.

The conduits described herein may include modified surfaces that prevent the channel from closing by reducing tissue growth into the passageway. The modified surfaces may also prevent the conduit from being ejected from the channel as the wound heals. The surfaces of the conduit may be modified, for example, by depositing a bioactive substance or medicine onto the exterior surface of the conduit. The bioactive substance may be disposed on, for example, portions of the tissue barrier or the hold-down members.

The bioactive substances are intended to interact with the tissue of the surgically created channels. These substances may interact with the tissue in a number of ways. They may, for example, accelerate wound healing such that the tissue grows around the exterior surface of the conduit and then stops growing; encourage growth of the epithelial or endothelial cells; inhibit wound healing such that the injury site (e.g., the channel or opening) does not heal leaving the injury site open; and/or inhibit infection (e.g., reduce bacteria) such that excessive wound healing does not occur which may lead to excessive tissue growth at the channel thereby blocking the passageway. However, the foregoing statements are not intended to limit the present invention and there may be other explanations why certain bioactive substances have various therapeutic uses in the lung tissue. Again, the bioactive substances are intended to prevent the implant from being ejected as well as prevent the lung tissue from filling or otherwise blocking the passageway of the conduit.

A variety of bioactive substances may be used with the devices described herein. Examples of bioactive substances include, but are not limited to, pyrolitic carbon, titanium-nitride-oxide, paclitaxel, fibrinogen, collagen, thrombin, phosphorylcholine, heparin, rapamycin, radioactive 188Re and 32P, silver nitrate, dactinomycin, sirolimus, cell adhesion peptide. Again, other substances may be used with the conduits such as those substances which affect the wound healing response (or rate) of injured lung tissue.

A cross section of a conduit 300 having a modified surface is shown in FIG. 3S. In particular, the conduit 300 comprises an inner frame layer or ribs 380 which define a passageway 381 for air to flow through. Coaxially surrounding the frame 380 is a tissue barrier 330. Additionally a visualization coating 384 is disposed on the tissue barrier 330. The visualization coating 384 is deposited as described above. A bioactive substance 386 is deposited on the visualization layer either directly or via a binding layer as described below. In this manner, the bioactive substance is disposed on an exterior surface of the conduit and contacts tissue when the device is deployed in a channel. However, it is contemplated that additional layers may be added such as, for example, an additional silicone layer over the visualization layer.

Also the order of the layers may be different than that described above. For example, the visualization layer may be disposed over the bioactive layer. Also, not all coatings and materials shown in FIG. 3S are necessary to carry out the present invention. For instance, the bioactive substances in some cases may be deposited directly on the open-frame 380.

The bioactive layer may also serve as the visualization coating or tissue barrier in some instances. For example, silicone and one or more bioactive substances may be mixed together and disposed on the conduit as a single coating. The single integral layer may serve both to physically and chemically prevent tissue from filling the conduit's passageway. It may also be visually apparent during a procedure.

Additionally, the bioactive substances may be deposited on the exterior surface of the conduit evenly or in discrete (intermittent) amounts. The thickness of the coatings may be uniform or the thickness may vary across certain regions of the conduit. This may provide higher therapeutic doses corresponding to certain regions of the injury site. For example, it may be desirable to provide a higher concentration of a bioactive substance near the ends of the conduit rather than in the center section.

The bioactive coatings may be selectively applied by spraying the bioactive substance onto uncovered regions of the conduit. For example, the bioactive substances may be disposed on at least a portion of the tissue barrier or the open-frame (or mesh) structure itself. The substances may also be applied by dipping, painting, printing, and any other method for depositing a substance onto the conduit surface. Additionally, binding materials may be applied to the exterior surface of the conduit upon which the bioactive agents may be deposited. Cross-linked polymers and or biodegradable polymers such as, for example, chondroitin sulfate, collagen and gelatin may be applied to the exterior surface of the conduit prior to depositing the bioactive substances. Additionally, the exterior surface of the conduit may be treated via etching processes or with electrical charge to encourage binding of the bioactive substances to the conduit.

Again, the bioactive substances also serve to reduce or impede tissue growth into the conduit's passageway. In this manner, the conduits maintain the patency of channels surgically created in the lung airways allowing air to pass therethrough.

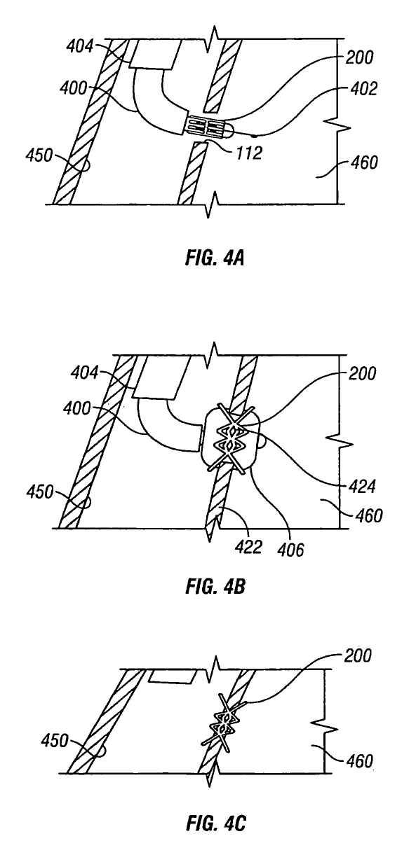

FIGS. 4A-4C illustrate a way to deploy a conduit in a channel. Referring to FIG. 4A, a delivery device 400 is loaded with a conduit 200. An access device 404 (e.g., an endoscope, a bronchoscope, or other device) may optionally be used to place the delivery device 400 into a collateral channel 112. A guide wire 402 may be used to place the delivery device 400 into the collateral channel 112. The guide wire 402 may be a conventional guide-wire or it may simply be comprised of a super-elastic material. The use of a guide wire is optional as the invention contemplates placement of the conduit 200 using only the delivery device 400.

FIG. 4A also illustrates articulation (or bending) of the deliver device 400 to access the collateral channel 112. However, the invention also contemplates articulation of the access device 404. The access device 404 may be articulated such that the delivery device 400 may advance straight into the collateral channel 112. Accordingly, the delivery device 400 may exit straight from the access device 404 or it may be articulated into the opening.

FIG. 4B illustrates deployment of the conduit 200. In particular, balloon member 406 is shown in an expanded state resulting in (1.) the conduit's center section being radially expanded and (2.) the conduit's extension members being outwardly deflected such that opposing extension members sandwich portions of the tissue wall 422. Diametric-control members 424 are also shown in this figure. The diametric or center-control segments limit the center section's radial expansion. In this manner, conduit 200 is securely placed in the channel to maintain a passageway through the airway wall 422.

FIG. 4C illustrates the deployed conduit 200 once the delivery device 400 is removed from the site.

It should be noted that deployment of conduits is not limited to that shown in FIGS. 4A-4C, instead, other means may be used to deploy the conduit. For example, spring-loaded or shape memory features may be actuated by mechanical or thermal release and unlocking methods. Additionally, mechanical wedges, lever-type devices, scissors-jack devices, open chest surgical placement and other techniques may be used to deploy the conduit. Again, the conduit 200 may be comprised of an elastic or super-elastic material which is restrained in a reduced profile for deployment and expands to its deployed state upon mechanical actuator or release.

In use, the conduit 200 is deployed with the distal side towards the parenchymal tissue 460 while the proximal side remains adjacent or in the airway 450. Of course, where the proximal and distal extension members are identical, the conduit may be deployed with either side towards the parenchymal tissue.

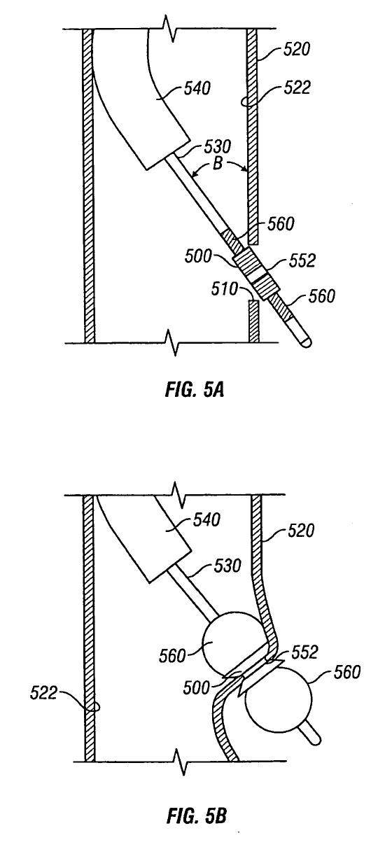

FIGS. 5A-5B illustrate another example of deploying a conduit 500 in a channel 510 (or opening) created in a tissue wall 520. Referring to FIG. 5A, a delivery tool 530 carrying a deployable conduit 500 is inserted into the channel 510. The delivery tool 530 is extended straight from an access catheter 540 such that the delivery tool forms an angle (B) with the tissue wall 520. It is to be understood that while the tissue wall of airway 522 is shown as being thin and well defined, the present invention may be utilized to maintain the patency of channels and openings which have less well defined boundaries. The delivery tool is further manipulated until the conduit is properly positioned which is determined by, for example, observing the position of a visualization mark 552 on the conduit relative to the opening of the channel 510.

FIG. 5B illustrates enlarging and securing the conduit in the channel using an expandable member or balloon 560. The balloon 560 may be radially expanded using fluid (gas or liquid) pressure to deploy the conduit 500. The balloon may have a cylindrical shape (or another shape such as an hourglass shape) when expanded to 1.) expand the center section and 2.) deflect the proximal and distal sections of the conduit such that the conduit is secured to the tissue wall 520. During this deployment step, the tissue wall 520 may distort or bend to some degree but when the delivery tool is removed, the elasticity of the tissue tends to return the tissue wall to its initial shape. Accordingly, the conduits disclosed herein may be deployed either perpendicular to (or non-perpendicular to ) the tissue wall.

A medical kit for improving gaseous flow within a diseased lung may include a conduit, a hole-making device (e.g., a needle or radio-frequency energy ablation/cutting catheter), a deployment device and/or a detection device. Examples of such methods and devices are described in U.S. patent application Ser. No. 09/633,651, filed on Aug. 7, 2000; U.S. patent application Ser. Nos. 09/947,144, 09/946,706, and 09/947,126 all filed on Sep. 4, 2001 each of which is incorporated by reference in its entirety. The kit may further contain a power supply, such as an RF generator, or a Doppler controller which generates and analyzes the signals used in the detection devices. The kit may include these components either singly or in combination.

The kit of the present invention may also contain instructions teaching the use of any device of the present invention, or teaching any of the methods of the present invention. The instructions may actually be physically provided in the kit, or it may be on the covering, e.g., lidstock, of the kit. Furthermore, the kit may also comprise a bronchoscope, or guide-member (such as a guide-wire), or other such device facilitating performance of any of the inventive procedures described herein. All the components of the kit may be provided sterile and in a sterile container such as a pouch or tray. Sterile barriers are desirable to minimize the chances of contamination prior to use.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. It is also contemplated that combinations of the above described embodiments/variations or combinations of the specific aspects of the above described embodiments/variations are within the scope of this disclosure.

1. A conduit for bronchoscopic placement within a channel created in lung tissue comprising:
a frame structure having a radially expandable center section, a proximal and distal sections located at a first and second ends of the center section;
a tissue barrier located about the frame structure, where the tissue barrier prevents tissue from growing into the frame structure; and
a visualization mark being visually identifiable from the remainder of the tissue barrier or frame structure, the visualization mark assisting in placement of the conduit relative to the channel.

2. The conduit of claim 1, further comprising at least one center control-segment attached to a portion of the center section, the center control member having a folded shape that unfolds upon radial expansion of the center section and restricts further expansion of the center section beyond a maximum profile, where the proximal and distal may expand beyond the maximum profile such that when expanded the frame structure is non-cylindrical

3. The implant of claim 1, where the tissue barrier comprises a polymeric material.

4. The implant of claim 3, where the frame structure comprises a plurality of members forming a mesh having a plurality of interstices

5. The implant of claim 3, where the polymeric material comprises a material selected from the group consisting of silicone, polyurethane, PET, PTFE, and expanded PTFE.

6. The implant of claim 3, wherein said tissue barrier is located about an interior of said center section.

7. The implant of claim 3, wherein the tissue barrier covers the mid portion, the tissue barrier further covers a proximal portion of the frame structure such that the distal portion of the frame structure remains uncovered.

8. The implant of claim 3, where the tissue barrier forms a first layer on the frame structure, the implant further comprising a second visible layer at least partially covering said first layer.

9. The implant of claim 1, where the visualization mark covers only a portion of the tissue barrier.

10. The implant of claim 1, where the visualization mark is disposed about the tissue barrier corresponding to the mid portion.

11. The implant of claim 1, where the visualization mark is disposed about the tissue barrier corresponding to only one of the proximal portion or the distal portion of the implant.

12. The implant of claim 1, where the visualization mark comprises a visible coating on the implant.

13. The implant of claim 1, where the visualization mark comprises a biocompatible polymer.

14. The implant of claim 1, where the visualization mark comprises a stripe circumferentially disposed about at least a portion the frame structure.

15. The implant of claim 1, further comprising a bioactive substance disposed on at least a portion of the tissue barrier.

16. The implant of claim 15, wherein said bioactive substance is selected from the group consisting of pyrolitic carbon, titanium-nitride-oxide, paclitaxel, fibrinogen, collagen, thrombin, phosphorylcholine, heparin, rapamycin, radioactive 188Re and 32P, silver nitrate, dactinomycin, sirolimus, cell adhesion peptide.

17. The implant of claim 2, where the folded shape of the center-control segment is selected from an arcuate shape, a semi-circular shape, an arcuate, an annular and a v-shape.

18. The implant of claim 17, wherein said at least one center-control segment is reinforced with at least one ancillary center-control segment.

19. The implant of claim 17, wherein said center-control segment is made of an elastic material.

20. The implant of claim 19, wherein said biocompatible polymer has a white color.

21. The implant of claim 1, where the mid portion comprises an inelastic material such that the mid portion does not automatically radially expand when unconstrained.

22. The implant of claim 1, where the mid portion comprises an elastic material and the mid portion automatically radially expands when unconstrained.

23. The implant of claim 1, where the frame structure comprises a material selected from the group of implantable materials consisting of polypropylene, PTFE, nylon, stainless steel, titanium, titanium alloy, MP35N, and nitinol.
